(19) United States
(12) Patent Application Publication
(10) Pub. No.: US 2022/0339456 A1
(43) Pub. Date: Oct. 27, 2022

(54) Treatment device to denature viral molecules in the human body by shattering their electrostatic viral fields by bombarding them with energy tuned at extremely high frequency electromagnetic waves

(71) Applicant: Richard Keen, Stamford, CT (US)

(72) Inventor: Richard Keen, Stamford, CT (US)

(21) Appl. No.: 17/239,938

(22) Filed: Apr. 26, 2021

(51) Int. Cl. A61N 1/40; A61L 2/00

(52) U.S. Cl. CPC A61N 1/40; A61L 2/0064

(57) ABSTRACT

This “in vivo” medical treatment/diagnostic invention has the “intended use” of bombarding the family of coronavirus cells (and COVID-19 and variants) with an energy beam at a tuned frequency to cause them to resonant at a turbulent velocity the outer shell shatters causing their destruction. A Treatment Probe is advanced through the mouth into the windpipe, then an energy beam of extremely high frequency electromagnetic waves causes the cells to react by oscillating at a turbulence sufficient to shatter their electrostatic viral fields thus denaturing these viral cells The remaining active cells are attack by the body's immune system. Repeating treatment sessions stimulate the body's immune system to denatures/destroys the family of coronavirus cells.

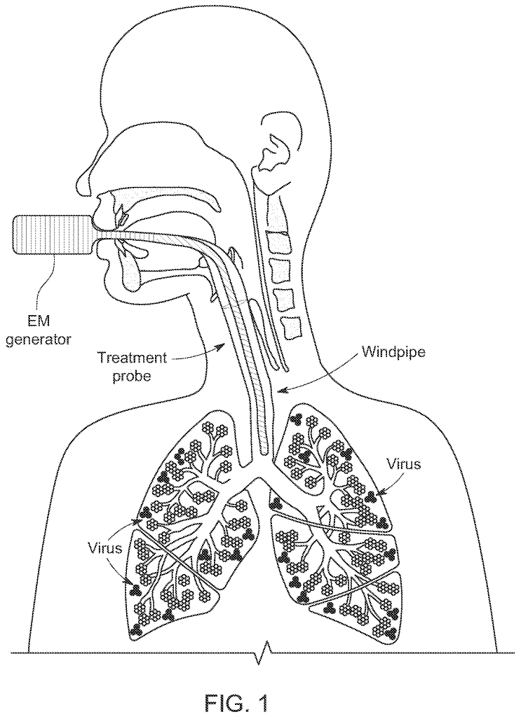

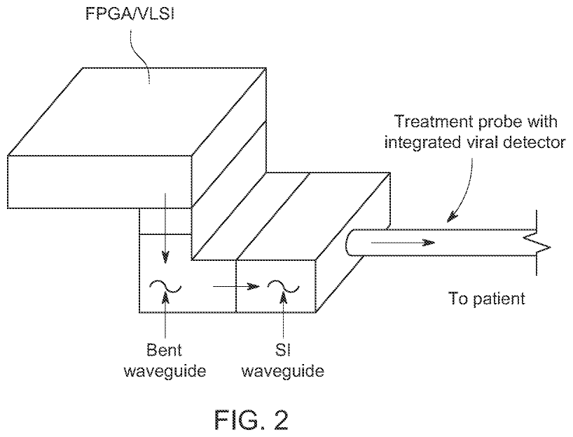

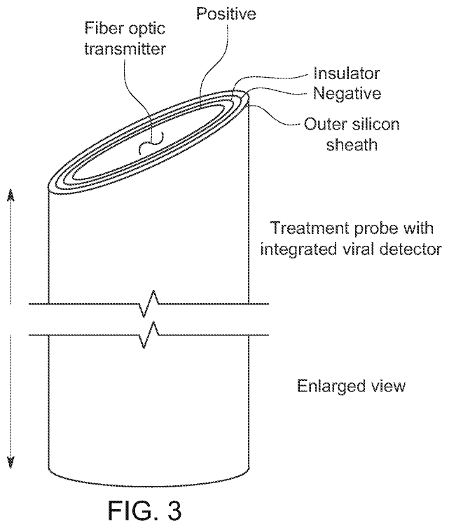

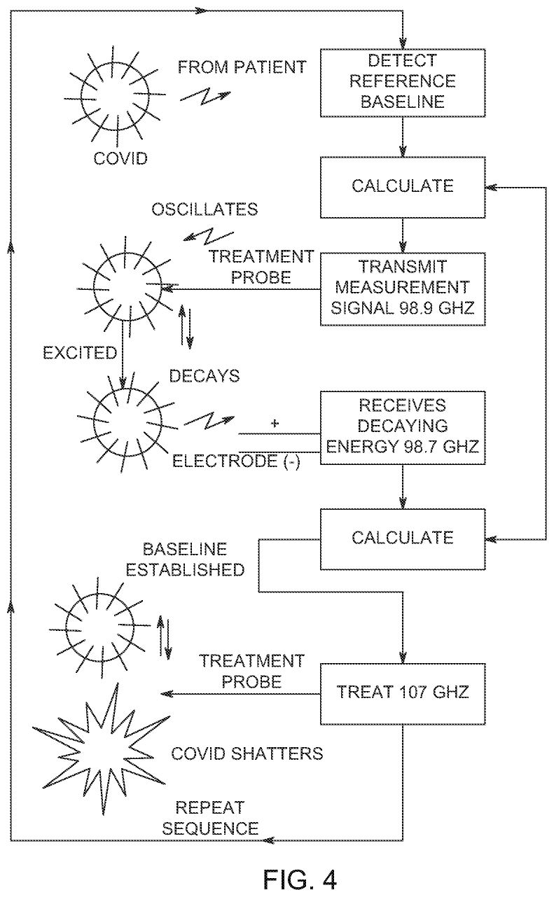

Table of Contents

| number | Title | page # |
|---|---|---|
| | TITLE | 2 |
| | PATENT DECLATION | 2 |
| | CROSS REFERENCE TO RELATED APPLICATIONS | 2 |
| | FEDERALLY SPONSORED RESEARCH | 2 |
| | SEQUENCE LISTING, ETC ON CD | 2 |
| | BACKGROUND OF THE INVENTION | 3 |
| | NEED FOR THIS INVENTION | 3 |
| | Description of Related Art | 5 |
| | DESCRIPTION OF RELATED ART | 5 |
| | BRIEF SUMMARY OF THE INVENTION | 6 |
| | Stimulating the immune system | 7 |
| | BACKGROUND OF THE INVENTION | 8 |
| | Technological characteristics | 8 |
| | DETAILED DESCRIPTION OF THE INVENTION | 9 |
| | Science Elements | 9 |
| | Energy Transfer Characteristics | 10 |
| | Description Of The Destructive Mechanism | 11 |
| | Power and Frequency | 12 |
| | Maximum Safe Power | 13 |
| | Safe Radiation Properties | 14 |
| | Hardware Design | 14 |
| | EM Generator Elements | 15 |
| | Diagnostic Mode | 16 |
| | Treatment Session Description | 17 |
| | Treatment/Diagnostic Sequencing | 18 |
| [031] | BRIEF DESCRIPTION OF THE DRAWING | 20 |
| | FIG. 1. Description | 20 |
| | FIG. 2. Description | 21 |
| | FIG. 3. Description | 21 |
| | FIG. 4. Description | 22 |
| | GLOSSARY OF TERMS | 22-25 |
| | ABSTRACT OF THE DISCLOSURE | 26 |
| | Drawing Document | 27-30 |
| | Claims Document | 31-32 |

### CROSS REFERENCE TO RELATED APPLICATIONS

Patent No. 63/101,959 describes a companion invention by the same inventor that uses this treatment device (63/101,2391) to achieve a specific outcome claimed in Patent No. 63/101,959. This treatment device is necessary to achieve the outcome that is claimed in 63/101,959. Currently the Provisional Patent No. 63/101,959 will be filed as a Utility Patent within the statuary deadline.

### PATENT DECLATION

This utility patent delineates a treatment/diagnostic device to treat patients by inserting a treatment probe into their windpipe that bombards a viral respiratory infection by using a high frequency radio frequency energy transmission to oscillate the electrically conductive viral cells to shatter from mechanical turbulence.

### FEDERALLY SPONSORED RESEARCH

Not applicable.

### SEQUENCE LISTING, ETC ON CD

Not applicable.

### BACKGROUND OF THE INVENTION

This invention relates to medical treatment devices in general and, more particularly treatment devices to treat a particular range of infections where normally pharmaceutical preparations are commonly relied on. In fact the use of this device is so unusual that it is almost considered ridiculous in medical circles—since such a device or a treatment procedure is not studied in medical schools. Conventional medical practice dictates if something other than a pharmaceutical preparation is to be used to treat this malady—then that treatment device that claims to accomplish the same remedy is viewed as fringe treatment outside the normal range of medical consideration.

### NEED FOR THIS INVENTION

Refer to Patent No. 63/101,959. The hope for the development of a vaccine for COVID-19 filled the airways and was a highly desirous objective world-wide in 2020 & 2021. Billions of dollars were devoted to the effort. The vaccine was viewed as a critical element needed to cause the return to normal life before the COVID-19 pandemic. This invention embodies all the properties of a treatment device to kill the virus and the outcome after a series of treatments as a vaccine to stimulate the immune system like a vaccine stimulates the immune system. The invention kills or destroys a quantity of the total viral load in a patient. At every session, some of the virus is destroyed and some of the viral cells-remain. Not all the viral cells are killed in one treatment session. The remaining infection can be treated and controlled where the patients benefits by an improvement in health and reduced level of the virus remains but the patient is not threatened by the infection. The residual amount of the viral infection can be managed for the patient's own immune system to attack and destroy it. This action has the same outcome as the biological function as a vaccine.

This is an en enviable action caused as a biological response of the immune system. So any method to reduce the infectious behavior of the COVID-19 virus to restore the health of the patient soothe patient's immune system can challenge the injection cause the same outcome as a vaccine.

This has the the superior benefit of treating a patient at any stage of infection whether the symptoms are minor or sever.

Since the human immune has a known action to invading infections, when a treatment (whether biological preparation of an electromagnetic beam) lessen the presence of the infection, the immune acts the same to defeat the infection. A significant advantage of this treatment over a vaccine is NO SIDE EFFECTS and the treatments can be repeated at any interval without biological consequences.

### DESCRIPTION OF RELATED ART

The field of medical devices for treatment of humans and animals has experienced an enormous expansion of devices and techniques. These developments have been enabled in part by advances in technological capabilities. Decades ago, ultra high frequencies were uncommon (and were well below 1 Giga-HERTZ). These frequencies were reserved for special purposes in large bulky equipment. Now, frequencies above 10 Giga-HERTZ are used routinely in a multitude of devices. The latest cellular phones (5G) work 24 to 52 GHZ. That was unobtainable a few decades ago. With the proliferation of these technologies, medical devices can now feature designs that were unheard of a few short years ago. This range of frequencies are referred to as the “micro-millimeter” spectrum. Now, medical treatment devices that can operate at higher frequencies can also enjoy the benefit of the same higher reliability and lower cost as cellular phones.

As part of these new capabilities, there has been an increase in the capability of components and testing capabilities that necessarily are required to accompany the higher frequencies. So, the higher frequencies that can be used are possible, as well as better components and methods. So, the stage is set for the possibilities for high performance electronically controlled treatment devices.

It should be noted that in many realms of medicine, the use of treatment devices have become accepted and normal. This is especially true with ambulatory treatment devices.

An example of this is where radiation is used to destroy cancer cells. Radiation treatment offers a remedy often where no other treatment is as effective. XXX This a denature viral molecules in the human body by shattering their electrostatic viral fields by bombarding them with energy tuned at extremely high frequency electromagnetic waves

The present invention generally comprises a new approach to introducing new medical technology to medical practitioners.

### BRIEF SUMMARY OF THE INVENTION

This patent describes a treatment and diagnostic device that generally comprises a method and apparatus for delivering medical treatment to a patient by a device that is operated by a medical practitioner on a patient infected by the COVID-19 or a virus in the coronavirus family. A significant feature of this invention and a compelling attribute is that this device destroys the infection once the patient is infected and the treatment session can be repeated until the patient no longer present symptoms. This device is designed to be an “in vivo” medical treatment whose “intended use” is to deliver an energy beam at a tuned frequency to cause viral molecules to resonant at a turbulence to cause their destruction.

The principal action of this patent is the device functions by emitting a tuned beam of radio frequency energy from a probe that is inserted inside a patient's windpipe. The energy beam acts on the viral infection of COVID-19 (or other viral derivatives) by bombarding the outer, spherical, electrically conductive shell to cause a mechanical oscillation that shatters the outer shell causing the viral properties to be destroyed.

Stimulating the Immune System.

The outcome of the use of this device, offers features not possible with a vaccine.

First, unlike a vaccine there are no unintended side effects which are always a risk with a biological preparation.

Second, a vaccine must be administered before a patient is exposed to COVID-19, once the infection occurs, vaccines are not effective. This device can be used before or after infection occurs.

Third, this mechanism is completely different than a vaccine, it simply destroys a percentage of the viral load for each treatment session.

Fourth, treatments can be repeated at frequent intervals without adverse biological affects.

Fifth, treatment can be administrated at any stage of infection.

Each treatment reduces the viral load for the immune system to overcome.

### BACKGROUND OF THE INVENTION

This device destroy virus cells in an infected patient by treating the virus in the windpipe with a beam of RF energy that shatters and denatures the conductive shell of the virus by the action of vibrating/oscillating/bombarding the cell. [0008] The foundation of is invention is based on a scientific phenomenal that the shell of the viral molecule is electrically conductive and has a native “static-type” electrical charge. When this charge is excited by an external energy wave (high frequency RF) the cell is forced to react. This reaction causes the cell shell to “oscillate or resonant”. That is a mechanical reaction. If the cell can be caused to mechanically react in an velocity or turbulence to a sufficient level, the turbulence can cause the protective shell to crack/fracture or become denatured to cause the destruction of the viral molecule.

Technological Characteristics

Device emits an energy beam with sufficient electromagnetic properties to form a tuned electric circuit with the cells of a enveloped RNA coronavirus.

This is analogous to a “parallel electric resonant circuit” where the viral cell absorbs maximum power at a resonant frequency causing the cell to oscillate to a point fracture.

The energy beam has electromagnetic properties with sufficient properties to form a bioelectric “parallel resonant” circuit where the outer conductive shell of the virus is caused to oscillation by absorbing addition energy at a tuned frequency. This oscillation causes a turbulence leading to the protective shell of the enveloped RNA viruses to fracture. When fractured, the internal elements of the cell “leak out” of their encapsulated environment and the cellular mechanism of the virus cannot continue functioning, thus apoptosis or cell death occurs.

This Treatment device denatures viral molecules in the human body by shattering their electrostatic, viral field by bombarding the cell with electrical energy that is tuned at “extremely high frequency electromagnetic waves”. The electrically conductive shell adsorbs this energy causing a transfer of energy from electrical energy into mechanical energy. The cell must dissipate this additional mechanical energy and does so by vibrating. If the input electrical energy can be raised to a threshold level so intense that the mechanical motion is constrained and the cell can no longer dissipate the added energy by vibrating then the additional energy causes the cell to shatter.

### DETAILED DESCRIPTION OF THE INVENTION

Science Elements

A “viral destructive energy” is applied to the treatment probe and bombard the viral field causing mechanical oscillation to “fracture” the statically charged viral molecule. The “fracture” is the result of resonating turbulence on the statically charged corona virus molecule.

Energy Transfer Characteristics

When the transmitted signal is electrically coupled to the electrically active viral shell, the excess energy is adsorbed by the outer electrically conductive shell according to Maxwell's theorems of predicted actions. The excess energy at the viral shell must be dissipated. This energy is converted from electrical energy to mechanical energy causing the outer shell of the virus cell to oscillate at a velocity necessary to cause a fracture or shattering of the outer viral shell. The exact mathematical pattern is designed to excite the viral cell to cause mechanical oscillation. This oscillation period persists for a duration in time necessary to cause the encapsulated outer coronavirus (COVID-19 and variants) spherical conductive shell to fracture or shatter.

This energy beam delivers micro millimeter radio transmission field that has a frequency pattern that outputs a unique power level that is designed to electrically couple with the electrical properties of the electro conductive spheres of the nearby coronavirus (COVID-19 and variants).

The energy is maintained for a period of time sufficient to kill/destroy/denature these coronavirus (COVID-19 and variants) shells to cause the cell to cease functioning as a virus.

Description of the Destructive Mechanism

By bombarding virus molecules with energy tuned to cause resonant oscillation, the virus molecules oscillate into an un-sustainable turbulence and then shutters—denaturing the viral molecule (causing its death).

Such a bombardment of energy is accomplished by placing a unique treatment probe to deliver the energy only after measuring the ambient viral infection.

A unique process using a Doppler type measurement combined with a computer algorithm allows for a dosage to be established for each treatment session. The probe emits an ultra high frequency radio energy pattern that causes the conductive outer protective shell of the covid19 virus cell (which is known to be electrically conductive) to cause sympathetic surface oscillation that would CRACK the outer shell of the virus and “de-nature” the cell. The cell would be adsorbed as a harmless substance by the body.

The Treatment Probe energy output signal and the electrical properties of the viral shell act in concert together to form an electrical “tank circuit”. Where these two entities functions as a parallel combination of a capacitor and inductor (conductive outer shell of vital cell). This is the “resonant circuit”—a circuit that absorbs maximum power at a particular frequency (called the resonant frequency).

The viral cells are spherical shaped objects. The mathematical formula that describes the energy pattern necessary to oscillate the spherical cells to vibrate to destruction is a TRADE SECRET.

The source of the destructive energy beam is emitted from the Treatment Probe. The

Treatment Probe is a SI waveguide. It is moved near the target (viral cells). The biological media that the viral cells are suspended do not function is the electrical circuit and is electrically non-reactive, but the mechanical elastically of the media represents an impedance to the adsorption of the energy and have a resistance to the motion of the cells.

These properties can be presented as coefficients in a polynomial equation. The value of each equation to “solve for” shall be the frequency and energy necessary to deliver a level of energy to cause excessive mechanical vibration to fracture the spherical viral cells. A primary variable to be solved for would be—the output RF frequency (high frequency radio frequency energy) or oscillation delivered to the SI waveguide.

Power and Frequency

1) Frequency. The frequency to achieve a tuned resonant to oscillate the cell is:

Frequency=J2×oscillation velocity/suspension media

2. The Power necessary to cause the destruction necessary energy at the output of the waveguide=source to target distance×time exposed oscillating X

Where

a) energy=source to target distance×time exposed oscillating X

b) J2=diameter of balls×wall thickness of ball/stiffness of balls

c) the suspension media adsorbs a percentage of the delivered energy

d) force necessary to cause oscillation

e) force necessary to cause ball to crack

f) distance of source to target

g) viscosity of suspension media

To fit into the patient's mouth, there is a molded disposable mouth piece with a feeder hole to accommodate the Treatment Probe.

The flexible steerable SI waveguide antenna & EM Generator is one encapsulated unit (where the length shall fit the patient as either a 9 inch or 13 inch length Treatment Probe.

Maximum Safe Power

The EM Generator presents a power at any surface that shall not exceed: 5.35 mW/cm² (Max average Power Density) for a treatment session with a specified time interval.

Safe Radiation Properties

The energy beam shall only produce non-ioning electromagnetic radiation that shall not contain sufficient energy per quantum to ionize atoms or molecules, where an electron from an atom or molecule is not interfered or dislodged from its intended properties.

Hardware Design

The device shown in FIG. 1 is highlighted in a hashed or dashed line to illustrate both the EM Generator and the Treatment Probe that is attached as an umbilical cord. The device consists of a EM generator fitted within a mouthpiece, to which a treatment probe is attached. In a treatment session, the Treat Probe is advanced to the lungs. This is the position in a patient for the treatment to occur.

The device is composed of the EM Generator with the attached Treatment Probe. The EM driver unit generates energy as a “tuned waveform” composed as a radio frequency and then applies/conducts this energy into the Treatment Probe.

This invention is a low cost assortment of disposable and re-usable components. (under $250) handheld device that might resemble a small VHF radio with a flexible antenna (7 mm diameter treatment probe). The EM Generator shall be controlled by an APP on smart phones and will communicates to the control driver.

Positioning treatment probe: This antenna/probe would be introduced as a steerable catheter into the mouth, down the throat or into the trachea depending on the threat. The Treatment Probe radiates this energy into the patient specified as the “Treatment mechanism”.

EM Generator Elements. The electromagnetic (RF frequency) EM Generator is composed of five subassemblies:

a) Interface circuitry—This circuitry receives a Bluetooth signal from the users smart phone and sets a series of parameters as configuration commands to the driver circuitry.

b) Frequency driver circuitry—This circuitry is composed of a FPGA/VLSI circuit board of chips that receives configuration commands and causes the driver circuitry to output the designed output power thru the antenna (Treatment Probe) to the patient. The electronic driver (FPGA/VLSI) is mated to the a bent waveguide and mechanically coupled to the SI waveguide to minimize the signal loss of the transmitted energy. The transmitted energy is outputted to the Treatment Probe as if it is a conventional radio antenna.

c) Bent waveguide—The output power from the driver is delivered to the bent waveguide. This changes the direction of the energy by 90 degrees to achieve the least propagation loss (compared to the straight loss) to conserve energy. With the direction changed, the energy is applied to the “antenna”.

d) Silicon waveguide—This is the Treatment Probe (SI waveguide). It is a circular tube about 12 MM in diameter and is flexible in a transverse direction. This probe is a single layer crystal silicon waveguide. It receives the energy from the driver (thru the bent waveguide) and radiates the energy to achieve the Treatment mechanism to the patient.

e) Power Subsystem—This circuitry receives external power to charge internal batteries to power subsystems a, b, c & d.

Diagnostic Mode

Refer to FIG. 4. In the diagnostic/measurement mode (REFERENCE), a detector receives the ambient electromagnetic signals at 98.7 GHZ through the electrodes in the treatment probe. Then the viral molecule is bombarded by a tuned emission (98.9 GHZ pulse train) that is transmitted as a source to achieve a “resonant excitation” in the target viral molecule. When the viral molecule that is excited and then the excitation energy declines back to its acquiescent energy, energy at an anticipated frequency is echoed back to the receiver.

That “echoed” energy is measured. The calculated resonance of 98.7 GHZ is detected and processed using a series of filters by a computer algorithm.

If a change in the energy from the reference to the received energy, then that difference is the response of the viral molecule to the transmitted source energy of 98.9 GHZ. This process is repeated at several variations until a stable value is established.

The measured values are filtered in an algorithm which computes the space factor in the spatial target, while the time factor defines a time limited pulse computed from the “delivered energy”. The ?TR? location corresponds to a unitary filter, ie the filter is “turned off” and thereby the treatment probe is broadcasting to the entire lung volume.

The Energy Beam is delivered to the Treatment Probe in either of two modes.

Destructive Mode. This electromagnetic field bombards the virus with a intensity set to destroy the viruses within the infected region.

The Diagnostic Mode is different than the Treatment Mode in that the bombardment level is at a non-destructive level. The lower energy has the purpose of eliciting a Doppler type reflection. In the diagnostic mode, the device bombards the virus with a non-destructive electro-magnetic energy for a designated time interval to measure the ambient level of infection. This electromagnetic field can be controlled to either excite the virus with a non-destructive field and detect an Doppler type echo to measure the quantity of the virus.

Treatment Session Description

In a treatment session, the Treat Probe is advanced to the lungs and proceeds as follows:

1) A “measurement energy” is delivered to the treatment probe to bias and tune the circuit to excite the electrostatic property of the virus. The response to this excitement is received by an electret receiver and filtered to calculate a measurement of the viral quantity or concentration. This excited response establishes the quantitative presence of the viral infection.

2) A “viral destructive energy” is applied to the treatment probe and bombard the viral field causing mechanical oscillation to “fracture” the statically charged viral molecule. The “fracture” is the result of resonating turbulence on the statically charged corona virus molecule.

The above sequence, Step 1 & Step 2 are repeated until the session is concluded

Treatment/Diagnostic Sequencing

The diagnostic sequence is a complex calculation ran by the processor of a smart phone to emit a non-destructive micro-millimeter RF emission to excite the viral colony. It does not target discrete cells. This is a Doppler type return echo to be detected by concentric charged dielectric material that has a quasi-permanent electric charge or dipole polarization within the Treatment probe.

This energy beam destroys/kills corona virus cells during a controlled treatment session and then switches from treatment mode to diagnostic mode. In Diagnostic Mode, the remaining viral quantity is measured. A calculation determines the residual amounts necessary to leave in the patient necessary to challenge the immune system and stimulate a natural recovery.

This diagnostic sequence occurs repeatedly to measure the field properties of the viral colony in the patient's respiratory system. This measurement indicates the quantification of the remaining infestation of a viral colony. The measurement causes the next treatment session.

To be effective, the energy must be effectively transferred without significant losses. This is achieved by inserting the Treatment Probe into the patient's Windpipe to achieve a proximity to the infection.

When the transmitted signal is electrically coupled to the electrically active viral shell, the excess energy is adsorbed by the shell and is converted from electrical to mechanical energy.

The excess electrical energy at the viral shell must be dissipated and is converted into mechanical energy according to Maxwell's theorems. This mechanical energy causes the shell to fracture. The exact mathematical pattern is designed to excite the viral cell to cause mechanical oscillation. This oscillation period persists for a duration in time necessary to cause the encapsulated outer coronavirus (COVID-19 and variants) spherical conductive shell to fracture or shatter.

Treatment session: The treatment session could be repeated as often as necessary based on the total viral load.

### BRIEF DESCRIPTION OF THE DRAWING

Refer to FIG. 1. The device is highlighted in a hash symbol to illustrate both the EM Generator and the Treatment Probe. The Treatment Probe is attached as an umbilical cord to the EM Generator. A smart phone will control the EM Generator and is not part of the invention. The device is shown inserted in a patient. The device consists of a EM generator fitted within a mouthpiece, to which a treatment probe is attached. The treatment probe is advanced through the mouth into the Windpipe. The anatomical distances necessary to achieve a effective treatment is not the subject of this patent.

Refer to FIG. 2. This drawing illustrates the EM Generator that is controlled by a smart phone (not part of this application). This electronic circuitry is composed of a FPGA/VLSI circuit board of chips that receives configuration commands (via Bluetooth) and causes the driver circuitry to output the designed output power into the Bent Waveguide. The Bent waveguide changes the direction of the energy by 90 degrees to achieve the least propagation loss (compared to the straight loss) to conserve energy. With the direction changed, the energy is coupled to the “antenna” (which is the Silicon waveguide). The Silicon waveguide or the Treatment Probe or the antenna is a flexible circular tube about 12 MM in diameter and is flexible in a transverse direction. This probe is a single layer crystal silicon waveguide. It receives the energy from the driver (thru the bent waveguide) and radiates the energy to achieve the Treatment mechanism to the patient.

Refer to FIG. 3. This is an enlarged view of the flexible SI tube to illustrate the detection or diagnostic elements of this invention. The Treatment Probe contains various layers of EM sensitive dielectrics. It is a multi-lumen tube with positive and negative dielectric electrodes. These electrodes are biased at extremely low levels to detect the presence of the virus. A quantitative measurement is developed to be used as a reference point to measure the presence of the virus.

Refer to FIG. 4. This block diagram illustrates the process to detect, develop a reference baseline of the quantity of the viral cells present, treat the virus (destroy) and then verify a reduction in the quantity has occurred. It is unknowable without exhaustive testing the point where any time period is associated with the destruction process and subsequent cellular decay or cell death.

Refer to FIG. 4. Efficacy Evaluation. In the evaluation mode, the device detects if a reduction has occurred in the quantity of the virus. This process is initiated by bombarding the virus cells with a REFERENCE non-destructive electro-magnetic energy for a designated time interval, then the mode is changed and the detectors receive an echo from the stimulation beam. to measure the ambient level of infection. The process alternates between the REFERENCE mode and the measurement mode. The evaluation continues during a session until a reduction in the quantity of the virus. is measured. The treatment can be repeated by multiple sessions to destroy and track the viruses simultaneously.

The REFERENCE energy is delivered to the treatment probe and tuned to excite the electrostatic property of the virus. The response (echo) to this excitement is received by an electret receiver and filtered to calculate a measurement of the viral quantity or concentration. This excited response establishes the quantitative presence of the viral infection.

### GLOSSARY OF TERMS

In describing the invention, the following terms that are used are defined:

Vaccine. A vaccine is a biological preparation that provides active acquired immunity to a particular infectious disease. A vaccine typically contains an agent that resembles a disease-causing microorganism and is often made from weakened or killed forms of the microbe, its toxins, or one of its surface proteins.

Viral cells or viral molecules. Any reference in this patent to “cells or viral cells or molecules” applies to the definition of the outer conductive spherical envelope or shell in RNA coronavirus or the COVID-19 virus or any of the multiple variants. This outer shell is described in science to be electrically conductive with electrostatic field properties.

Properties of the Virus Outer Shell. This shell is characterized as a spherical protective shell that encapsulates the viral cell. This shell is electrically conductive and has an electrostatic field.

Fracture/Shatter/Denatured Virus Cell. This phenomena occurs when the mechanical pressure on the outer shell reaches a crescendo or peak intensity where the outer shell fractures.

Windpipe or trachea. A large cylindrical membranous tube reinforced by rings of cartilage, extending from the larynx to the bronchial tubes and conveying air to and from the lungs; the windpipe Treatment mechanism. The device transmits an energy beam with sufficient electromagnetic properties to form a tuned electric circuit with the cells of a enveloped RNA coronavirus. This energy causes the outer conductive shell to oscillate at a level of turbulence to fracture the surface of the cells. When the envelope or shell is fractured the cellular mechanism of the virus cannot continue functioning, thus apoptosis or cell death occurs.

Oscillate. A mechanical motion or vibration to oscillate or swing back and forth in a pattern.

Resonant. A prolonged synchronous vibration such as a mechanical motion especially in a hollow body tending to reinforce or prolong synchronous vibrations.

FPGA/VLSI. This is an electronic circuit production technique to achieve the necessary computer logic and driver circuits

FPGA. This term applies to “Field Programmable Gate Array”. That defines ten thousand to more than a million logic gates with programmable interconnection.

VLSI. This term applies to an electronic circuit production technique “Very large-scale integration” to create an integrated circuit (IC) by combining millions of MOS transistors onto a single chip. This enables a complex semiconductor and telecommunication technologies to be developed.

SI waveguide. This is a construction technique to use a silicone substrate as a high frequency waveguide to construct a flexible antenna.

Antenna. The antenna and the Treatment Probe are the same entity. This is a hybrid construction using silicon as a substrate to form an antenna to conduct radio frequency for transmission.

Treatment mechanism. The antenna radiates the energy from the driver and transmits it to the patient's anatomy.

These claims define this invention:

1. An in vivo medical treatment whose “intended use” is to deliver an energy beam at a tuned frequency to cause viral molecules to resonant at a turbulence to cause their destruction.

2. This energy beam delivers micro millimeter radio transmission field having a frequency pattern at a power level as to electrically couple with the electrical properties of nearby coronavirus (COVID-19 and variants) (COVID-19) electro conductive spheres.

3. When electrically coupled, is transmitted energy causes a viral cells to adsorb this transmitted energy according to Maxwell's theorems of predicted actions of coupled electric and magnetic fields interacting as energy waves.

4. The Treatment Probe is inserted into the patient's Windpipe to achieve a proximity to the infection and to optimize the exposure to the infestation of a viral colony.

5. The energy field excites these coronavirus (COVID-19 and variants) electro conductive spheres with such a pattern and design to cause these coronavirus (COVID-19 and variants) spheres to absorb this electrical energy and become in such an excited state that the viral cell must dissipate the received energy by mechanically oscillating or vibrating.

6. This oscillation persists for a duration in time necessary to cause the encapsulated outer coronavirus (COVID-19 and variants) spherical conductive shell to fracture or shatter.

7. The constant energy presented by the Treatment Probe is sufficient to kill/destroy/denature these coronavirus (COVID-19 and variants) shells causing the cell to cease functioning as a virus.

8. A diagnostic measurement sequence where energy is emitted to react with the viral molecules and the reaction is measured to yield a value that indicates the quantification of the remaining infestation of a viral colony.

9. This invention applies to a complex calculation system ran by a computer to emit a controlled microwave emission process that results in the destruction of viral infections in the throat and lungs.

10. After an elapsed period of time a reference RF signal is transmitted to excite the remaining coronavirus (COVID-19 and variants) cells to cause a Doppler type return echo to be detected by concentric charged dielectric material that has a quasi-permanent electric charge or dipole polarization within the Treatment probe. This measure the remaining quantity of the virus.

11. This energy beam destroys/kills corona virus cells during a controlled treatment session and then switches modes to measures the remaining viral quantity. A calculation determines the residual amounts necessary to leave in the patient necessary to challenge the immune system and stimulate a natural recovery.
